(19) United States
(12) Patent Application Publication
(10) Pub. No.: US 2026/0354977 A1
(43) Pub. Date: Nov. 12, 2026

(54) HYDRAULIC PENILE EXTRACORPOREAL IMPLANT

(71) Applicant: Oleksandr Volodymyrovych KNIGAVKO, Kharkiv (UA)

(72) Inventor: Oleksandr Volodymyrovych KNIGAVKO, Kharkiv (UA)

(21) Appl. No.: 19/326,858

(22) Filed: Sep. 12, 2025

(30) Foreign Application Priority Data
May 9, 2025 (UA) U 2025 02185

(51) Int. Cl. A61F 2/26; A61F 2/74

(52) U.S. Cl. CPC A61F 2/26; A61F 2/74

(57) ABSTRACT

The invention relates to medicine, specifically to surgery, urology, and andrology. The objective is achieved in that, in the known hydraulic penile extracorporeal implant, which includes a fluid reservoir, a pump, a tube, and a valve according to the invention, the implant consists of four components, wherein the first component is an expander consisting of six semicylinders lying on one membrane in a crescent shape, connected proximally and distally; in frontal projection on the penis, the expander has a crescent shape; upon inflation, the expander increases in three directions: from 2 mm to 7 mm in thickness, from 6 cm to 8 cm in width, and from 7.5 cm to 15 cm in length; the expander may be 6 cm in length and may increase up to 12 cm; the expander may be 9 cm and may increase up to 18 cm; the expander shell is made of medical-grade silicone and reinforced mesh with elastic longitudinal and dense oblique-transverse fibers; the outer silicone layer of the expander is hydrophilic; for fixation, the distal part of the expander has holes in the reinforced membrane; the second element is a stabilizer, which is a triangular silicone implant measuring 2.5 cm by 3.5 cm with rounded edges; the expander has two protruding rods of 5 mm, and the stabilizer has two corresponding recesses for fixation and connection; the stabilizer may be used in two sizes—2.5 cm and 4 cm along the upper part; the pump with the valve has a working volume of 4 ml, and when pressed, transfers fluid from the reservoir to the expander, causing it to increase in three directions; the pump has a blind nozzle; the fourth element is a reservoir with a valve with a total volume of 120 ml; the expander, the pump, and the reservoir are connected by two reinforced tubes with an internal diameter of 1 mm, with a connector.

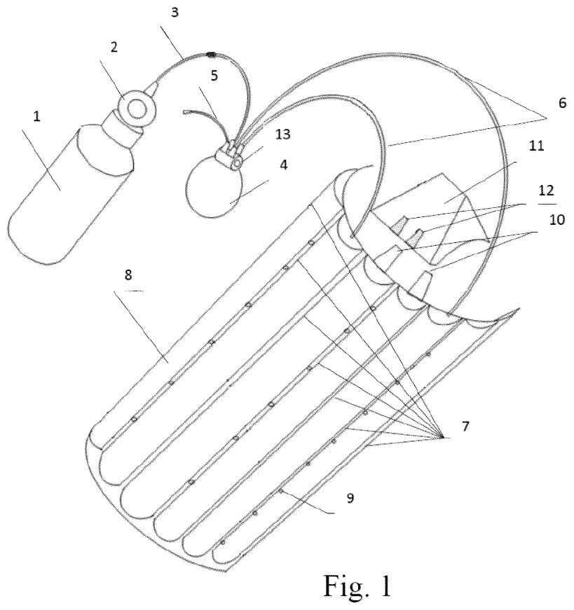

The invention relates to medicine, specifically to surgery, urology, and andrology, and can be used for stretching the skin and tissues of the penis during prosthesis inflation, to stimulate their growth for subsequent plastic, reconstructive, and restorative surgery of the damaged penis, as well as to provide additional rigidity to the penis in cases of erectile dysfunction.

The closest analogue is a penile erectile system (U.S. Pat. No. 4,523,584, IPC A61F2/26. Penile erectile system/Medical Eng Corp, Yachia Daniel, Lynch Henry W.—No. US 19830472155; filed Mar. 4, 1983; published Jun. 18, 1985), which includes a sleeve made of biocompatible material that has at least one sealed chamber intended for implantation around the shaft of the penis under the penile skin, between the skin and the shaft, a fluid reservoir that creates pressure, and a pump for transferring fluid under pressure from the reservoir to the chamber. The system also includes a tube connecting the reservoir and pump to the pressurized chamber, and a valve for controlling fluid flow into and out of the chamber. The pressure-increasing pump serves as both a reservoir and a pump.

The disadvantages of the closest analogue are that this device is designed to improve erection, is only a two-component system (extracorporeal implant and pump-reservoir), and does not stretch well in length. Its main shortcomings include the possibility of prolapse of the prosthesis through the skin due to the subcoronal implantation method, as well as insufficient elongation of this prosthesis during artificial erection.

These shortcomings necessitated the development of the hydraulic penile extracorporeal implant.

The goal of the invention is the development of a hydraulic penile extracorporeal implant. The developers named this device the Hydraulic Penile Extracorporeal Implant “Penogrande.”

The objective is achieved in that, in the known hydraulic penile extracorporeal implant, which includes a fluid reservoir, a pump, a tube, and a valve according to the invention, the implant consists of four components, wherein the first component is an expander consisting of six semicylinders lying on one membrane in a crescent shape, connected proximally and distally; in frontal projection on the penis, the expander has a crescent shape; upon inflation, the expander increases in three directions: from 2 mm to 7 mm in thickness, from 6 cm to 8 cm in width, and from 7.5 cm to 15 cm in length. The expander may be 6 cm in length and may increase up to 12 cm; the expander may also be 9 cm and may increase up to 18 cm; the expander shell is made of medical-grade silicone and reinforced mesh with elastic longitudinal and dense oblique-transverse fibers; the outer silicone layer of the expander is hydrophilic; for fixation, the distal part of the expander has holes in the reinforced membrane; the second element is a stabilizer, which is a triangular silicone implant measuring 2.5 cm by 3.5 cm with rounded edges; the expander has two protruding rods of 5 mm, and the stabilizer has two corresponding recesses for fixation and connection; the stabilizer may be used in two sizes—2.5 cm and 4 cm along the upper part; the pump with the valve has a working volume of 4 ml, and when pressed, transfers fluid from the reservoir to the expander, causing it to increase in three directions; the pump has a blind nozzle; the fourth element is a reservoir with a valve with a total volume of 120 ml; the expander, the pump, and the reservoir are connected by two reinforced tubes with an internal diameter of 1 mm, with a connector.

The technical effect of the invention, namely the development of a hydraulic penile extracorporeal implant, is due to the synergy of the device's structural components. The hydraulic penile extracorporeal implant can be used as a device to improve erection, which is especially important for patients with partially preserved erectile function who do not wish to have an intracorporeal prosthesis implanted and damage the cavernous tissue inside the corpora cavernosa of the penis. The hydraulic penile extracorporeal implant can be used for the following purposes: stretching the tissues of the penis prior to reconstructive surgery; lengthening and thickening of the penis in individuals with micropenis or acquired post-traumatic or postoperative penile defects; improving erection in individuals with partially preserved erectile function with good arterial inflow but loss of rigidity due to venous leakage or psychological issues; restoration of erection and partial length/thickness of the penis in individuals for whom intracavernous prostheses cannot be installed due to cavernous fibrosis or absence of the glans and distal parts of the corpora cavernosa; improving the aesthetic appearance of the penis in cases of corporal trauma.

The hydraulic penile extracorporeal implant as claimed is illustrated graphically in the figure, where: reservoir—1, valve—2, connector—3, pump—4, blind nozzle—5, two reinforced tubes—6, six semicylinders—7, expander—8, holes in the reinforced membrane—9, two protruding rods—10, stabilizer—11, two indentations—12, valve 13.

The hydraulic penile extracorporeal implant consists of four parts. The first component is the expander 8, which consists of six semicylinders 7 lying on a single membrane in the shape of a crescent and connected to each other proximally and distally. In the frontal view on the penis, the expander 8 has a crescent shape and subcutaneously wraps over the penis from 7 to 5 o'clock on a conceptual clock face. When the expander 8 is inflated, the expander 8 increases in three directions: from 2 mm to 7 mm in thickness, from 6 cm to 8 cm in width, and from 7.5 cm to 15 cm in length. The expander may be 6 cm in length and may increase to 12 cm. The expander may be 9 cm in length and may increase to 18 cm. This uneven increase of the expander is achieved through the special use of various materials: a shell made of medical-grade silicone and a reinforced mesh with elastic longitudinal and dense oblique-transverse fibers, which allow elongation when the expander 8 is filled with fluid. The outer silicone layer of the expander 8 is hydrophilic, allowing it to be soaked in an antibiotic and hemostatic solution before implantation to reduce the risk of infection and hematoma. The expander 8 is fixed distally beneath the glans of the penis via the holes in the reinforced membrane 9 and sutured with absorbable threads (3/0) to the lateral part of the corpora cavernosa.

The second component is a stabilizer 11, a triangular silicone implant measuring 2.5 cm by 3.5 cm with rounded edges. It is implanted through an infrapubic incision between the pubic bone and the corpora cavernosa at the site of the severed suspensory ligament. The role of the stabilizer 11 is to hold and stabilize (prevent rotation or displacement of) the proximal part of the expander 8, which has two protruding rods 10 of 5 mm, and the stabilizer 11 has two matching indentations 12 for fixation and connection.

The third component is a pump 4 with a valve 13. The pump 4 has a working volume of 4 ml and, when pressed, transfers fluid from the reservoir 1 to the expander 8, causing it to increase in three directions. The pump 4 has a blind nozzle 5, whose main purpose is to allow fluid to be directed toward a future intracavernosal prosthesis, if needed.

The fourth component is a reservoir 1 with a valve 2, with a total volume of 120 ml, which is located in the pre-pubic space near the bladder.

The expander 8, the pump 4, and the reservoir 1 are connected by two reinforced tubes 6 with an internal diameter of 1 mm and a connector 3. When the pump 4 is pressed, fluid (saline solution) from the reservoir 1 is pumped into the expander 8, causing it to increase in thickness, width, and length, which results in stretching of the skin and internal tissues and a corresponding increase in the size of the penis, similar to a natural erection.

When the valve 2 is pressed, the fluid flows back in the opposite direction to the reservoir 1, and the expander 8 decreases in all three directions, wherein the penis also returns to its normal size.

The inflation and deflation of the expander 8 stretch the tissues of the penis, increasing its size without damaging the blood supply to the tissues.

A method of using the hydraulic penile extracorporeal implant:

Use of the implant is recommended for circumcised patients. If the patient has excess foreskin, it is better to remove it two months before the main implantation procedure. After thorough aseptic preparation of the male torso, and under spinal or general anesthesia, a 4 cm transverse incision or rhombus-shaped incision with 3 cm lateral limbs is made in the infrapubic area, followed by ligamentotomy and hemostasis. A No. 16 Foley catheter is inserted into the patient's urethra, then, dissection of the tissues between Buck's fascia and the subcutaneous fascia of the penis is performed with careful hemostasis. The internal tissues of the penis: corpora cavernosa with the urethra are everted into the wound and measured in a partially extended state, and said length corresponds to the combined length of the stabilizer and the uninflated expander. That is, the length of the stretched penis from the depth of the wound between the corpora cavernosa and the pubic bone to the glans penis should equal the sum of the expander and the stabilizer lengths.

The expander is available in three sizes: 6 cm, 7.5 cm, 9 cm. The stabilizer is available in two sizes: 2.5 cm, 4 cm (measured across the top). This allows for optimal sizing for each individual patient.

The stabilizer is fixed to the periosteum of the pubic bone using absorbable suture (2/0).

The distal end of the expander is fixed to the lateral surfaces of the corpora cavernosa using absorbable suture (2/0) in two rows on both the left and right sides.

The length compatibility between the penis and the expander with the silicone stabilizer is verified, afterward, the two locking elements (rod and indentation) between the expander and the stabilizer are engaged.

The pump with the valve is implanted subcutaneously in the scrotum between the testicles, in an anterior position, making it easily accessible for pressing through the skin.

The empty reservoir is implanted either through the upper part of the inguinal canal (right or left side), or via a muscle-sparing approach into the pre-pubic space.

The system is filled with fluid—regular saline solution: 120 ml into the reservoir, and 5 ml into the system, followed by connection with the connector.

The functionality of the expander is tested by pumping fluid from the reservoir into the system using the pump.

1. A hydraulic extracorporeal penile implant, which includes a fluid reservoir, a pump, a tube, and a valve, characterized in that the implant has an expander consisting of six semi-cylinders positioned on a single membrane in the shape of a crescent, interconnected at the proximal and distal ends; when inflated, the expander increases in three directions; the expander shell is made of medical-grade silicone and a reinforced mesh composed of elastic longitudinal fibers and dense diagonal-cross fibers; the outer silicone layer of the expander is hydrophilic; for fixation at the distal end, the expander contains holes in the reinforced membrane; the implant includes a triangular silicone stabilizer with rounded edges; the stabilizer has two indentations, while the expander has two protruding rods for fixation and connection; the implant includes a pump with a valve and a blind nozzle, and a reservoir with a valve, through which fluid is transferred to the expander when the pump is pressed; the expander, the pump, and the reservoir are connected by two reinforced tubes with a connector.
